(19) United States
(12) Patent Application Publication
(10) Pub. No.: US 2008/0160508 A1
(43) Pub. Date: Jul. 3, 2008

(54) ASSAY METHOD AND ASSAY KIT FOR VIRUS RESPIRATORY INFECTION

(75) Inventors: Takeshi IMOARAI, Kobe-shi (JP); Koji OCHIAI, Kakogawa-shi (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION, Kobe-shi (JP)

(21) Appl. No.: 11/958,962

(22) Filed: Dec. 18, 2007

(30) Foreign Application Priority Data
Dec. 28, 2006 (JP) JP 2006-353541

(51) Int. Cl. C12Q 1/70

(52) U.S. Cl. 435/5

(57) ABSTRACT

An assay method for virus respiratory infection, is provided with steps of: providing a first immunochromatographic test device for assaying a first virus respiratory infection, and a second immunochromatographic test device for assaying a second virus respiratory infection different from the first virus respiratory infection; preparing an assay sample by treating a biological sample with a sample treatment liquid; assaying a part of the assay sample by using the first test device; and assaying a part of the assay sample by using the second test device when the assay result with the first test device is negative.

### CROSS REFERENCE TO RELATED APPLICATION

This application claims the benefit of priority from Japanese Patent Application 2006-353541, filed on Dec. 28, 2006, the entire disclosure of which is incorporated herein by reference.

### FIELD OF THE INVENTION

The present invention relates to an assay method for virus respiratory infections by using a test device for immunochromatography.

### BACKGROUND

The cold syndrome is a disease affecting everyone, and the background of its patients and its infectious factors are varied. For the cold syndrome, there are 200 or more causative agents including subtype viruses, and the number of causative agents that can be inspected by clinicians is limited. Accordingly, it is difficult to grasp its clinical states and to diagnose its causal agent. Since recent development of antiviral agents for influenza, evidence-based infection control has been required, and it is becoming necessary to take viral infections into consideration, from the time of infection outbreak. Particularly in the influenza season in winter, the discrimination of influenza from other virus respiratory infections (adenovirus, respiratory syncytial virus (referred to hereinafter as RS virus)) showing influenza-like symptoms is considered important for patients and for risk management of patients' surroundings.

At present, the examinations of influenza, adenovirus, RS virus etc. are conducted using individual assay kits for the respective items. Consequently, when a patient with influenza-like symptoms is negative on influenza testing, a sample should be collected again from the patient in order to examine adenovirus or RS virus. The sample is collected from the nasal cavity or pharynx by using a cotton-tipped swab or the like, and such collection may be accompanied by a pain thus imposing a burden on the patient. This is significant particularly in pediatric medical examination.

JP-A 2000-292427 describes a test device for lateral flow-type immunoassay capable of detecting a plurality of viruses. This test device, as shown in FIG. 1, uses an anti-rotavirus antibody, an anti-calicivirus antibody, an anti-coronavirus antibody, an anti-adenovirus antibody and an anti-enterovirus antibody as dye-bound latex labeled antibodies, and has an anti-rotavirus antibody-immobilized site 1, an anti-calicivirus antibody-immobilized site 2, an anti-coronavirus antibody-immobilized site 3, an anti-adenovirus antibody-immobilized site 4 and an anti-enterovirus antibody-immobilized site 5. A measurement sample prepared from patient's feces can be added to a sample inlet to inspect infection with the above 5 viruses.

When the technique described in JP-A 2000-292427 supra is applied to virus respiratory infections, the problem of patient's burden accompanying multiple sample collection will be reduced. However, the test device in JP-A 2000-292427 supra is problematic in a higher per-piece cost of the test device due to use of antibodies against plural types of viruses. In the case of virus respiratory infections, influenza for example will often prevail from December to March, while RS virus will prevail from October to January. Though there are overlapping epidemic periods, a doctor, even upon strongly suspecting a patient of having influenza during the examination, should inevitably examine the plural types of viruses for the patient if the test device described in JP-A 2000-292427 supra is used, thus resulting in higher examination costs. JP-A 2000-292427 supra describes preparation of samples of feces to be examined, but does not contain any description of preparation of assay samples containing a highly viscous substance mucin, such as samples (aspirates from the nasal cavity, fluids wiped out of the nasal cavity and fluids wiped out of the pharynx) to be examined in virus respiratory infections.

### SUMMARY

The scope of the present invention is defined solely by the appended claims, and is not affected to any degree by the statements within this summary. An assay method for virus respiratory infection in a first aspect of the present invention is provided with steps of providing a first immunochromatographic test device for assaying a first virus respiratory infection, and a second immunochromatographic test device for assaying a second virus respiratory infection different from the first virus respiratory infection; preparing an assay sample by treating a biological sample with a sample treatment liquid; assaying a part of the assay sample by using the first test device; and assaying a part of the assay sample by using the second test device when the assay result with the first test device is negative.

An assay kit for virus respiratory infection in a second aspect of the present invention is provided with a sample treatment liquid for preparing an assay sample; a sample treatment container accommodating the sample treatment liquid; a first immunochromatographic test device for assaying a first virus respiratory infection; and a second immunochromatographic test device for assaying a second virus respiratory infection different from the first virus respiratory infection.

### BRIEF DESCRIPTION OF THE DRAWINGS

FIG. 1(a) shows a test device for assaying an influenza virus infection used in the embodiment of the present invention.

FIG. 1(b) shows a test device for assaying an RS virus infection used in the embodiment.

FIG. 1(c) shows a test device for assaying an adenovirus infection used in the embodiment.

FIG. 2(a) is a front view of a test container used in the embodiment of the present invention.

FIG. 2(b) is a plan view of the test container.

FIG. 2(c) is a side view of the test container.

FIG. 2(d) is an I-I sectional view of FIG. 2(a).

FIG. 2(e) is an II-II sectional view of FIG. 2(b).

FIG. 2(f) is an III-III sectional view of FIG. 2(b).

FIG. 3 is a view showing one example of a sample treatment container used in the embodiment of the present invention.

FIG. 4 is a view showing one example of the test device used in another embodiment.

FIG. 5 is a view showing one example of the test device used in another embodiment.

FIG. 6 is a view showing one example of the test device used in another embodiment.

### DETAILED DESCRIPTION OF THE EMBODIMENT

The biological sample serving as an object inspected in the assay method for virus respiratory infection in the embodiment of the present invention is an aspirate from the nasal cavity, a fluid wiped out of the nasal cavity, a fluid wiped out of the pharynx, a nasal discharge or the like, among which an aspirate from the nasal cavity, a fluid wiped out of the nasal cavity and a fluid wiped out of the pharynx are preferable from the viewpoint of accuracy in viral collection.

Pathogens causing virus respiratory infections include influenza A virus, influenza B virus, RS virus (RSV) and rhinovirus in the case of viral infections occurring mainly in winter, and pathogens causing infectious gastroenteritis in children include rotavirus, norovirus, adenovirus and astrovirus.

Hereinafter, the test device used in virus respiratory infection in the embodiments of the present invention is described by reference to the drawings. The structure shown in the drawings and the following description is set forth for illustrative purposes and not intended to limit the scope of the present invention.

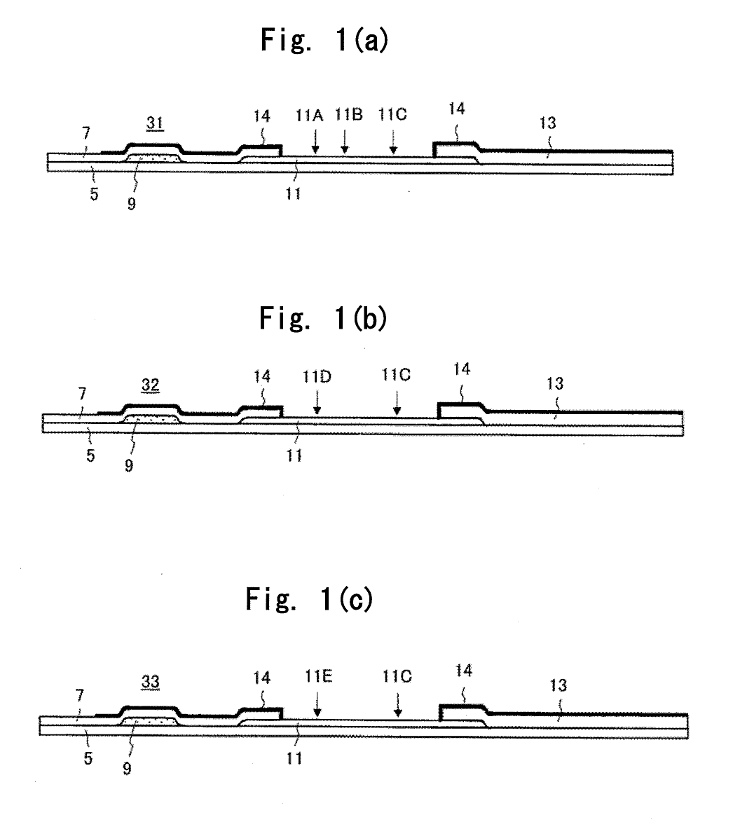

FIG. 1 is a sectional view of an immunochromatographic test device of lateral flow type, wherein (a) is a test device 31 for assaying an influenza virus infection, (b) is an immunochromatographic test device 32 for assaying an RS virus infection, and (c) is an immunochromatographic test device 33 for assaying an adenovirus infection.

As shown in FIG. 1(a), the test device 31 for assaying an influenza virus infection is provided, on a base material 5 consisting of a plastic plate having a pressure-sensitive layer thereon, with a sample addition member 7 consisting of a nonwoven rayon fabric, a label retaining member 9 consisting of a nonwoven glass fiber fabric, a chromatographic membrane carrier 11 consisting of porous nitrocellulose, an absorbent member 13 consisting of a nonwoven cellulose fabric, and a transparent seal 14 for covering the sample addition member 7 and the absorbent member 13 respectively as shown in the figure. The sample addition member 7 functions as a sample addition part dipped in a sample accommodated in a sample container 1. The label retaining member 9 is arranged in contact with the sample addition member 7 and functions as a label retaining part for retaining a label to be bound via antigen-antibody reaction to a substance to be detected in a sample. The chromatographic membrane carrier 11 is arranged in contact with the label retaining member 9 and has a judgment part on which an immobilization substance to be bound via antigen-antibody reaction to a substance to be detected is immobilized. The absorbent member 13 is arranged so as to contact with the chromatographic membrane carrier 11.

When the sample addition member 7 is dipped in a sample, the sample flows by capillary phenomenon through the label retaining member 9 and the chromatographic membrane carrier 11, thereby being developed to the absorbent member 13. On the chromatographic membrane carrier 11, a first judgment part 11A, a second judgment part 11B and a control part 11C are formed in the form of a line downwards in this order along the direction of development of the sample. A first labeled substance, a second labeled substance and a control labeled substance are retained in the label retaining member 9. An influenza A antibody (referred to hereinafter as “anti-Flu A antibody”), an influenza B antibody (referred to hereinafter as “anti-Flu B antibody”) and biotin are immobilized as immobilization substances on the first judgment part 11A, the second judgment part 11B and the control part 11C, respectively. The first labeled substance and the second labeled substance are respectively the anti-Flu A antibody and the anti-Flu B antibody, both of which have been labeled with blue latex particles, and the control labeled substance is avidin labeled with red latex particles. The anti-Flu A antibody and the anti-Flu B antibody will bind respectively to a first detection subject (influenza A virus) and a second detection subject (influenza B virus) (referred to hereinafter as “Flu A virus” and “Flu B virus” respectively) via antigen-antibody reaction.

For example, when Flu A virus is contained in a sample, the labeled anti-Flu A antibody in the label retaining member 9 recognizes, and binds via antigen-antibody reaction to, a predetermined site of the Flu A virus, to form a conjugate. Then, an anti-Flu A antibody in the chromatographic membrane carrier 11 recognizes, and binds to, a different site of the Flu A virus, thereby capturing the conjugate. This conjugate contains blue latex particles as label, so when the conjugate is captured, a blue line appears in the first judgment part 11A, whereby the Flu A virus can be visually detected.

Avidin is not captured by the anti-Flu A antibody and anti-Flu B antibody in the chromatographic membrane carrier 11, but binds specifically to biotin, and is thus captured by biotin immobilized on the control part 11C. Avidin has been labeled with red latex particles, so when the avidin is captured, a red line appears in the control part 11C, so it can be visually confirmed that the avidin has reached the control part 11C. The control part 11C is arranged downstream from the first judgment part 11A and the second judgment part 11B, and thus it can be confirmed by the occurrence of a red line that the sample has passed through the first judgment part 11A and the second judgment part 11B.

The test device 32 for assaying an RS virus infection shown in FIG. 1(b) has approximately the same constitution as in the above-mentioned test device 31 for assaying an influenza virus infection except for labeled substances retained in a judgment part and in a label retaining member 9. In FIG. 1(b), an anti-RS virus antibody is immobilized as an immobilization substance on the judgment part 11D. The labeled substances retained in the label retaining member 9 are an anti-RS virus antibody labeled with blue latex particles and a control labeling substance (avidin labeled with red latex particles). Like the test device 31, biotin is immobilized on a control part 11C. The anti-RS virus antibody will bind via antigen-antibody reaction to RS virus as a substance to be detected.

The test device 33 for assaying an adenovirus infection shown in FIG. 1(c) has approximately the same constitution as in the above-mentioned test device 32 for assaying an RS virus infection except for labeled substances retained in a judgment part and in a label retaining member 9. In FIG. 1(c), an anti-adenovirus antibody is immobilized as an immobilization substance on the judgment part 11E. The labeled substances retained in the label retaining member 9 are an anti-adenovirus antibody labeled with blue latex particles and a control labeling substance (avidin labeled with red latex particles). The anti-adenovirus antibody will bind via antigen-antibody reaction to adenovirus as a substance to be detected.

Hereinafter, the test container used in the assay method in the embodiment of the present invention is described.

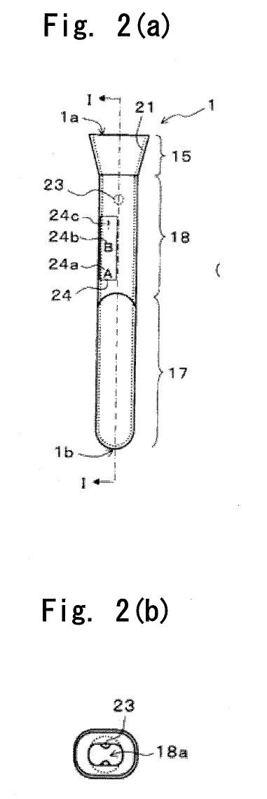

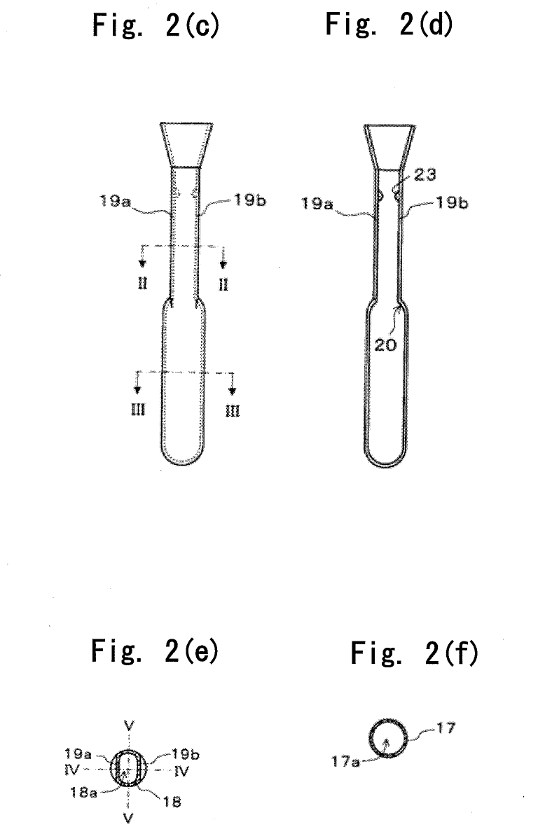

FIGS. 2(a), 2(b) and 2(c) are respectively a front view, a plan view and a side view of the test container 1, and FIG. 2(d) is an I-I sectional view of FIG. 2(a). FIG. 2(e) and FIG. 2(f) are respectively II-II and III-III sectional views of FIG. 2(b).

The test container 1 has a receiving part 15 having an opening 1a, a sample accommodating part 17 for accommodating a sample in bottom 1b, and an intermediate part 18 positioned between the receiving part 15 and the sample accommodating part 17.

The receiving part 15 is shaped such that the area of an inner section thereof perpendicular to the longer direction of the test container 1 (the direction of from the bottom 1b to the opening 1a of the test container 1, that is, the direction of the line I-I in FIG. 2 (a)) is increased towards the opening 1a. In this specification, the “inner section” means a section of the internal space of the test container 1. Unless otherwise specified, the “inner section” means an inner section of a plane perpendicular to the longer direction of the test container 1. Because the receiving part 15 has such shape, a sample can be easily introduced into the test container 1, and when the test container 1 falls down, a sample hardly falls out of the test container 1 because the longer direction of the test container 1 after falling is directed upward relative to the horizon. The fact that a sample hardly falls out of the test container 1 is described later in more detail.

By way of example, the side wall 21 of the receiving part 15 is tapered such that the area of an inner section of the receiving part 15 is increased towards the opening 1a.

As shown in FIGS. 2(e) and (f), the intermediate part 18 is shaped such that the area of the inner section 18a perpendicular to the longer direction of the test container 1 is smaller than the inner section 17a perpendicular to the longer direction of the sample accommodating part 17, and that the inner section 18a of the intermediate part 18 has such a long and thin shape that for example upon insertion of the test device 31 into the test container 1, the test device 31 can be prevented from turning round. The inner section 18a of the intermediate part 18 preferably has such a long and thin shape that upon insertion of the test device 31 into the test container 1, the test device 31 can be rotated within ±45° (more preferably within ±30°). The sign “±” refers to an angle upon clockwise and counterclockwise rotation respectively. Accordingly, “within ±45°” for example indicates that the test device can rotate clockwise at an angle within 45° and counterclockwise at an angle within 45°.

The intermediate part 18 has a first planar section 19a and a second planar section 19b opposed to the first planar section 19a. The first planar section 19a is positioned so as to correspond to the first and second judgment parts 11A and 11B of the test device 31 upon insertion of the test device 31 into the test container 1. When the test container 1 is transparent, the first and second judgment parts 11A and 11B are observed through the first planar section 19a that is so planar as to facilitate observation of the first and second judgment parts 11A and 11B without distorting an image of the first and second judgment parts 11A and 11B. The distance between the first and second planar sections 19a and 19b is shorter than the width of the test device 31 so that the first and second planar sections 19a and 19b can prevent the test device 31 from turning round in the test container 1.

The inner section 18a of the intermediate part 18 is made smaller in width than the inner section 17a of the sample accommodating part 17, in a direction perpendicular to the first and second planar sections 19a and 19b (in the direction of the line IV-IV in FIG. 2(e)). Accordingly, a step 20 has been formed between the sample accommodating part 17 and the intermediate part 18. When the test container 1 falls down, the step 20 functions in preventing a sample from falling out of the container. The inner section 18a of the intermediate part 18 is made equal in width to the inner section 17a of the sample accommodating part 17, in a direction (direction of the line V-V in FIG. 2(e)) parallel to the first and second planar sections 19a and 19b and perpendicular to the longer direction of the test container 1. In this direction too, the inner section 18a of the intermediate part 18 may be made smaller in width than the inner section 17a of the sample accommodating part 17 to form the step 20.

In the sample container 1, the inner section 18a of the intermediate part 18, along the whole of the longer direction thereof, is made smaller in area than the inner section 17a of the sample accommodating part 17, but in a part of the longer direction, the inner section 18a may be made smaller in area than the inner section 17a of the sample accommodating part 17.

The inner surface of the sample container 1 is provided with protrusions 23 for preventing the principal surface of the test device 31 (that is, the front face (face having the first and second judgment parts 11A, 11B etc. formed thereon) or the back face (face to which the base material 5 is exposed)) from adhering to the inner surface of the test container 1. The protrusions 23 shown in FIG. 2(a) to (f) are conically-shaped with its top rounded, but may be in other shapes such as sphere, cylinder, polygonal pyramid and polygonal cylinder. The top of the protrusion 23 may be sharp or rounded. In the test container 1, the protrusion 23 is arranged in each of the first and second planar sections 19a and 19b, in a position near to the receiving part 15, but the protrusion 23 may be arranged in another position, or two or more protrusions 23 may be arranged in each of the first and second planar sections 19a and 19b.

The test container 1 has marks 24a and 24b which in positions corresponding to the first and second judgment parts 11A and 11B of the test device 31, indicate the first and second judgment parts 11A and 11B of the test device 31 respectively, upon insertion of the test device 31 into the test container 1. The marks 24a and 24b on the test container 1 are “A” and “B”, respectively. The test container 1 has a mark 24c which in a position corresponding to the control part 11C of the test device 31, indicates the control part 11C of the test device 31 upon insertion of the test device 31 into the test container 1. The mark 24c on the test container 1 is “!”. The marks 24a to 24c indicate types of the first and second judgment parts 11A and 11B and the control part 11C.

The test container 1 described above is marked so as to correspond to the test device 31 for influenza virus assay, while the test container 1 for the test device 32 for RS virus assay is marked so as to correspond to the judgment part of the test device 32 for RS virus assay, and the test container 1 for the test device 33 for adenovirus assay is marked so as to correspond to the judgment part of the test device 33 for adenovirus assay.

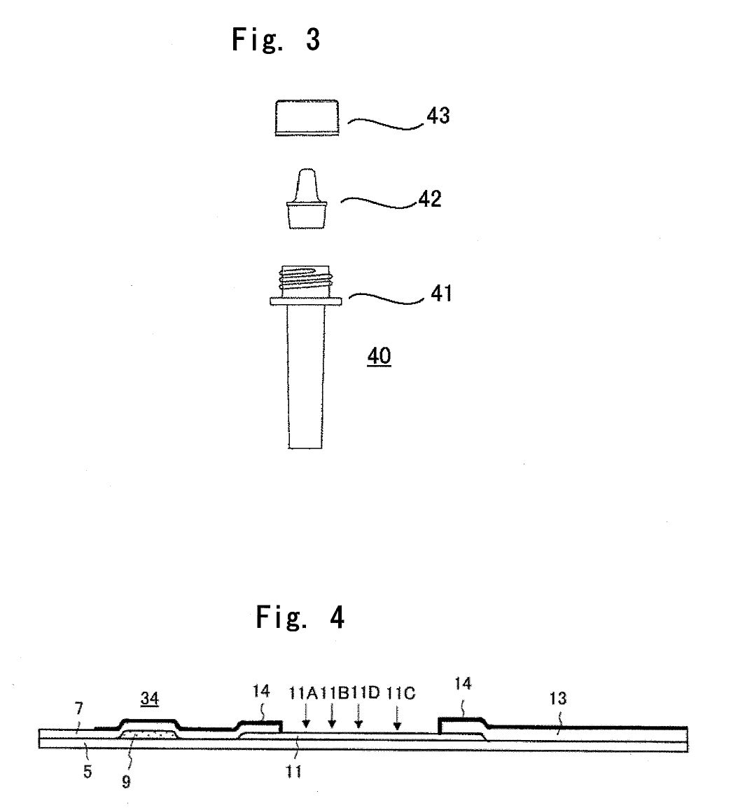

The sample treatment container 40 used in the assay method in the embodiment of the present invention is described. The sample treatment container 40 shown in FIG. 3 is composed of a plastic bottle 41, a nozzle 42 and a cap 43. The nozzle 42 is provided at its top with a sample discharge opening and equipped therein with a filter member.

The sample treatment container 40, when not used, accommodates a sample treatment liquid in the bottle 41 and stores the liquid in the bottle 41 whose opening is closed with the cap 43. Just before use, the cap 43 is opened, and a collected sample is added to, and mixed with, the sample treatment liquid in the bottle 41. Thereafter, the nozzle 42 instead of the cap 43 is fit in the opening of the bottle 41, and the sample (measurement sample) mixed with the sample treatment liquid passes through the filter member 44 and is fed from the sample discharge opening 46 to the sample container 1. The test device 31 is inserted into the sample container 1 accommodating the measurement sample such that the sample addition member 7 is positioned in the bottom of the sample container 1, to initiate the examination of influenza virus.

The filter member fitted to the inside of the nozzle 42 comprises a first glass fiber filter paper with a membrane pore diameter of 1.5 μm and a thickness of 0.4 mm, a second glass fiber filter paper with a membrane pore diameter of 23 μm and a thickness of 0.4 mm, and a nonwoven glass filter with a thickness of 0.7 mm laminated in this order. This filter member is fitted to the nozzle 42 such that the glass filter is placed at the side of the nozzle 42 attached to the bottle 41 and the first glass fiber filter paper at the side of the sample discharge opening. The filter member is not limited to this constitution, but the nonwoven glass filter is preferably used to remove viscous components in the sample, and one or two glass fiber filter papers are used in this nonwoven glass filter.

Then, the sample treatment liquid for a sample used in the assay method for virus respiratory infection in the embodiments of the present invention is described. The sample treatment liquid is preferably an aqueous solution containing a surfactant. This is because the surfactant makes openings in an outer skin of influenza virus through which an antigen protein in the virus can be transferred to the sample treatment liquid. As the surfactant, a nonionic surfactant and an amphoteric surfactant can be used.

Although the nonionic surfactant is not particularly limited, nonionic surfactants having polyoxyethylene can be preferably used, and more preferably nonionic surfactants having polyoxyethylene alkyl ether or nonionic surfactants having polyoxyethylene alkyl phenyl ether can be used. Specifically, it is preferable to use one or more members selected from the group consisting of polyoxyethylene alkyl phenyl ethers such as polyoxyethylene (9) octyl phenyl ether, polyoxyethylene (10) octyl phenyl ether and polyoxyethylene (9) nonyl phenyl ether, polyoxyethylene sorbitan fatty esters such as polyoxyethylene sorbitan monolaurate and polyoxyethylene sorbitan monooleate, a polyoxyethylene/polyoxypropylene copolymer, and a polyoxyethylene alkyl ether.

Although the amphoteric surfactant is not particularly limited, 3-[(3-cholamidopropyl)dimethylammonio]-1-propanesulfonate (CHAPS) or the like is preferably used. When the amount of the nonionic surfactant added to the sample treatment liquid is increased, the amphoteric surfactant may be used in combination therewith to improve the solubility and increase the storage stability of the sample treatment liquid.

The sample treatment liquid preferably contains a thiocyanic acid-based compound in order to prevent unspecific reaction. The thiocyanic acid-based compound is not particularly limited insofar as it is a water-soluble thiocyanic acid-based compound such as a thiocyanic acid ester or a thiocyanate, in addition to thiocyanic acid (NHCS). The constituent of thiocyanate includes inorganic bases including metals such as sodium, potassium etc. and organic base ammonium salts. The thiocyanate also includes hydrates and solvates of these salts. Specific examples include sodium thiocyanate, potassium thiocyanate, ammonium thiocyanate, guanidine thiocyanate, etc. among which potassium thiocyanate and guanidine thiocyanate are preferable.

The sample treatment liquid preferably contains a reducing agent to decrease the viscosity of highly viscous substances occurring in samples that are nasal discharges (aspirates from the nasal cavity, fluids wiped out of the nasal cavity) and fluids wiped out of the pharynx. The reducing agent is preferably a sulfur-containing reducing compound and includes, for example, mercaptoethylamine, mercaptoethylamine hydrochloride, mercaptoethanol, dithiothreitol, cysteine, N-acetyl-L cysteine, S-2 aminoethylisothiourea dihydrobromide, tris(2-carboxyethyl)phosphine, hydrosulfite salt, sulfite salt etc.

The sample treatment liquid may contain a chelating agent to suppress the activity of an enzyme decomposing an antigen protein or to reduce nonspecific reaction. The chelating agent can include, for example, ethylenediaminetetraacetic acid, 1,2-cyclohexanediaminetetraacetic acid, hexamethylenediaminetetraacetic acid, iminodiacetic acid, hydroxyethyliminodiacetic acid, 1,3-diaminopropan-2-oltetraacetic acid, diethylenetriaminepentaacetic acid, ethylenediaminediacetic acid, ethylenediaminediacetic acid dipropionic acid, ethylenebis(oxyethylenenitrilo)tetraacetic acid, ethylenediamine-tetrakis(methylenephosphonic acid), ethylenediaminedipropionic acid, hydroxyethylethylenediaminetriacetic acid, N-(2-hydroxylethyl)ethylenediaminetriacetic acid, nitrilotriacetic acid, nitrilotripropionic acid, nitrilotris(methylenephosponic acid), 2(hydroxyethyl)glycine and 1,2-diaminopropanetetraacetic acid, as well as salts thereof.

Alkali metal ions may be contained in the sample treatment liquid. The alkali metal ions can be exemplified by lithium⁺ (Li⁺), sodium⁺ (Na⁺), potassium⁺ (K⁺), rubidium⁺ (Rb⁺), cesium⁺ (Cs⁺) and francium⁺ (Fr⁺), among which sodium and potassium can be preferably used. Alkali metal ions can be used alone or as a mixture of two or more thereof. Compounds capable of generating such alkali metal ions are not particularly limited, and for example, a mixture of one or more members selected from the group consisting of sodium chloride, potassium chloride, sodium hydroxide, potassium hydroxide, EDTA sodium salt, and sodium azide can be used. By adding alkali metal ions, nonspecific reaction can be suppressed. The content of alkali metal ions is 0.3 to 2.0 M, preferably 0.4 to 1.5 M, more preferably 0.45 M to 1.0 M.

The sample treatment liquid preferably contains a buffer, and examples of the buffer can include Good buffers such as MES, Bis-Tris, ADA, PIPES, ACES, MOPSO, BES, MOPS, TES, HEPES, DIPSO, TAPSO, POPSO, HEPPSO, EPPS, Tricine, Bicine, TAPS, CHES, CAPSO and CAPS, among which ADA, PIPES, ACES, MOPSO, BES, MOPS, TES, HEPES, DIPSO, TAPSO, POPSO, HEPPSO and EPPS are preferable, and PIPES, ACES, MOPSO, BES, MOPS, TES and HEPES are more preferable. The pH of the sample treatment liquid is 5 to 10, preferably 5.5 to 9.0, more preferably 6.0 to 8.0.

Measurement sample prepared by pre-treating sample such as aspirates from the nasal cavity, fluids wiped out of the nasal cavity and fluids wiped out of the pharynx, with the sample treatment liquid described above, can be preferably assayed with the test devices for plural types of virus respiratory infections. Accordingly, viral infections can be examined successively starting from the viral infection most likely to occur depending on the epidemic period, thus preventing assay costs from increasing. Even when the first sample is negative on virus infection testing so another virus infection assay is conducted, a measurement sample prepared from the sample collected in the previous virus infection assay can be used, and thus examination of plural types of viral infections can be carried out without increasing a burden on the patient.

In the embodiment described above, the test devices for examining influenza often prevailing from December to March, RS virus prevailing from October to January, and adenovirus prevailing in every season have been illustrated, but are not limited thereto. A measurement sample prepared with the above-mentioned sample treatment liquid can be examined suitably with the test device for another prevailing virus respiratory infection. For example in the season when both influenza and RS virus prevail, a test device 34 that can examine influenza A virus, influenza B virus and RS virus simultaneously, as shown in FIG. 34, may be used, and when a sample is negative with the test device 34, the test device for assaying another infection such as adenovirus infection may be used to assay the sample.

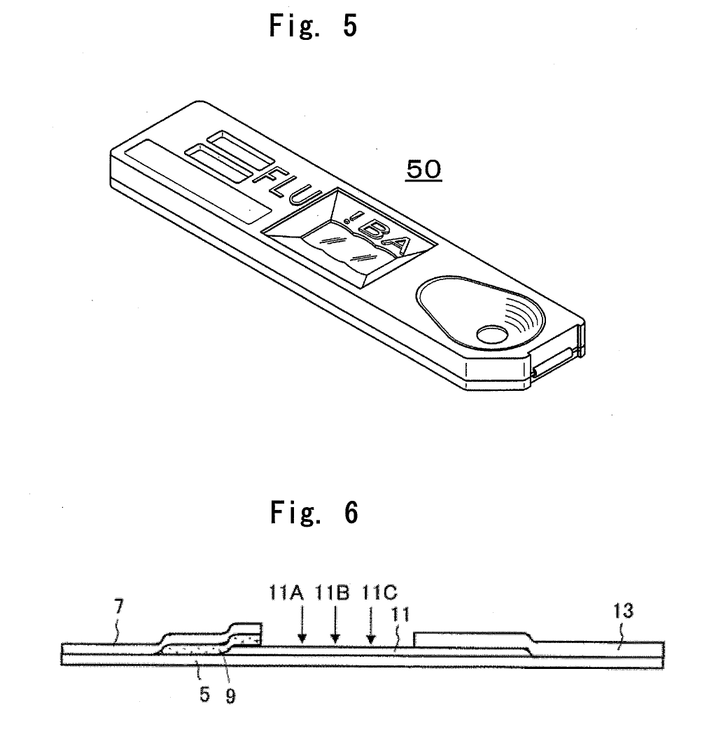

In the embodiment described above, the test devices 31 to 33 and the test container 1 are used in assay, but a test device 31, 32 or 33 may be used by accommodating it in a case 50 as shown in FIG. 5. In this case, use of the test container 1 is not necessary for assay, and a measurement sample may be dropped directly from the sample treatment container 40 onto the sample addition part of the case 50. As the test device in the embodiment described above, a test device having the structure shown in FIG. 6 may be used. In the embodiment described above, the test device of lateral flow type has been illustrated, but a test device of flow-through type can also be used. Alternatively, the test device of different type can be used for each of virus respiratory infections.

In the test devices 31 to 34 in the embodiment described above, the base material 5 is for suitably arranging the above-mentioned members such as the sample addition member 7 and the label retaining member 9, and can make use of various materials such as paper and glass besides plastics. The sample addition member 7 can be made up of various materials such as cotton, glass fiber or cellulose fiber besides rayon. The label retaining member 9 can be made up of various materials such as cellulose fiber besides glass fiber. The chromatographic membrane carrier 11 can be made up of various materials including not only nitrocellulose but also nylon (for example, nylon modified by introducing amino groups optionally substituted with a carboxyl group and an alkyl group), polyvinylidene difluoride (PVDF), and cellulose acetate. The absorbent member 13 can be made up of various materials such as glass fiber besides cellulose. The sample addition member 7, the label retaining member 9, the chromatographic membrane carrier 11 and the absorbent member 13 can use various structures which besides a nonwoven fabric and a porous body, can develop a sample by capillary phenomenon.

An assay kit for virus respiratory infections may also be provided. This assay kit comprises, for example, the test device for influenza virus assay, the test device for RS virus assay, and the sample treatment container accommodating the sample treatment liquid. The assay kit may further comprise the test device for adenovirus assay. The assay kit may further comprise the test container.

### EXAMPLES

Hereinafter, the present invention is described in more detail by reference to the Examples. However, the present invention is limited to the Examples.

### (1) Preparation of a Test Device 31 for Influenza Virus Assay

A chromatographic membrane carrier 11 and a label retaining member 9 were prepared according to the following method, and then used to prepare a test device 31 for influenza virus assay.

### (1-1) Preparation Sample of a Chromatographic Membrane Carrier 11

As shown in FIG. 1(a), an anti-influenza A monoclonal antibody diluted at a concentration of 2.0 mg/mL with a phosphate buffer, pH 7.0, an anti-influenza B monoclonal antibody diluted at a concentration of 1.5 mg/mL with a phosphate buffer, pH 7.0, and biotin-bound BSA (bovine serum albumin) diluted at a concentration of 1.0 mg/mL with a phosphate buffer, pH 7.0, were applied with an antibody applicator (BioDot Ltd.) onto a first judgment part 11A, a second judgment part 11B and a control part 11C of a chromatographic membrane carrier 11 made of a nitrocellulose membrane, and then dried at 50° C. for 30 minutes.

After drying, the chromatographic membrane carrier 11 was blocked by dipping in a blocking solution (BSA-containing phosphate buffer, pH 7.0). After blocking, the carrier 11 was washed with a wash (SDS-containing phosphate buffer, pH 7.0) and dried at 40° C. for 120 minutes to prepare the chromatographic membrane carrier 11.

### (1-2) Preparation of a Label Retaining Member 9

Blue colored polystyrene latex particles (particle diameter 0.3 μm) were sensitized with an anti-influenza A monoclonal antibody and then suspended in a dispersing buffer solution (phosphate buffer, pH 7.0, containing BSA and sucrose), to prepare anti-influenza A monoclonal antibody-sensitized latex particles.

Blue colored polystyrene latex particles (particle diameter 0.3 μm) were sensitized with an anti-influenza B monoclonal antibody and then suspended in a dispersing buffer solution (phosphate buffer, pH 7.0, containing BSA and sucrose), to prepare anti-influenza B monoclonal antibody-sensitized latex particles.

Red colored polystyrene latex particles (particle diameter 0.19 μm) were sensitized with streptavidin and then suspended in a dispersing buffer solution (phosphate buffer, pH 7.0, containing BSA and sucrose), to prepare streptavidin-sensitized latex particles.

The anti-influenza A monoclonal antibody-sensitized latex particles, the influenza B monoclonal antibody-sensitized latex particles, and the streptavidin-sensitized latex particles were mixed with one another, and the resulting mixed latex was added to a glass fiber pad (832 μL/300 mm×5 mm) and then dried in a vacuum drier to prepare a label retaining member 9.

### (1-3) Attachment of Each Member to a Base Material and Cutting of the Base Material

As shown in FIG. 1(a), the chromatographic membrane carrier 11 prepared in (1-1) above, the label retaining member 9 prepared in (1-2) above, a sample addition member 7 consisting of a nonwoven fabric (rayon), and an absorbent member 13 consisting of a nonwoven fabric (cellulose) were attached to a base material 5 consisting of a backing sheet. Then, a transparent sheet 14 was attached so as to cover the sample addition member 7 and absorbent member 13 as shown in the figure. Finally, the specimen was cut into pieces of 5 mm in width by a cutting machine (BioDot Ltd.) to prepare a test device 31 for influenza virus assay.

### (2) Preparation Sample of a Test Device 32 for RS Virus Assay

### (2-1) Preparation of a Chromatographic Membrane Carrier 11

As shown in FIG. 1(b), an anti-RS virus monoclonal antibody (reacting with an F protein of RS virus) diluted at a concentration of 2.0 mg/mL with a phosphate buffer, pH 7.0, and biotin-bound BSA (bovine serum albumin) diluted at a concentration of 1.0 mg/mL with a phosphate buffer, pH 7.0 were applied with an antibody applicator (BioDot Ltd.) onto a judgment part 11D and a control part 11C of a chromatographic membrane carrier 11 made of a nitrocellulose membrane, and then dried at 50° C. for 30 minutes.

After drying, the chromatographic membrane carrier 11 was blocked by dipping in a blocking solution (BSA-containing phosphate buffer, pH 7.0). After blocking, the carrier 11 was washed with a wash (SDS-containing phosphate buffer, pH 7.0) and dried at 40° C. for 120 minutes to prepare the chromatographic membrane carrier 11.

### (2-2) Preparation of a Label Retaining Member 9

Blue colored polystyrene latex particles (particle diameter 0.3 μm) were sensitized with an anti-RS virus monoclonal antibody (reacting with both RS virus A and B) and then suspended in a dispersing buffer solution (phosphate buffer, pH 7.0, containing BSA and sucrose), to prepare anti-RS virus monoclonal antibody-sensitized latex particles.

Streptavidin-sensitized latex particles were prepared in the same manner as in (1-2) above.

The anti-RS virus monoclonal antibody-sensitized latex particles were mixed with the streptavidin-sensitized latex particles, and the resulting mixed latex was added to a glass fiber pad (832 μL/300 mm×5 mm) and then dried in a vacuum drier to prepare a label retaining member 9.

### (2-3) Attachment of Each Member to a Base Material and Cutting of the Base Material

As shown in FIG. 1(b), the chromatographic membrane carrier 11 prepared in (2-1) above, the label retaining member 9 prepared in 2-2 above, a sample addition member 7 consisting of a nonwoven fabric (rayon), and an absorbent member 13 consisting of a nonwoven fabric (cellulose) were attached to a base material 5 consisting of a backing sheet. Then, a transparent sheet 14 was attached so as to cover the sample addition member 7 and absorbent member 13 as shown in the figure. Finally, the specimen was cut into pieces of 5 mm in width by a cutting machine (BioDot Ltd.) to prepare a test device 32 for RS virus assay.

### (3) Preparation of a Test Device 33 for Adenovirus Assay

### (3-1) Preparation Sample of a Chromatographic Membrane Carrier 11

As shown in FIG. 1(c), an anti-adenovirus monoclonal antibody (mouse IgG monoclonal antibody) diluted at a concentration of 2.0 mg/mL with a phosphate buffer, pH 7.0, and biotin-bound BSA (bovine serum albumin) diluted at a concentration of 1.0 mg/mL with a phosphate buffer, pH 7.0 were applied with an antibody applicator (BioDot Ltd.) onto a judgment part 11E and a control part 11C of a chromatographic membrane carrier 11 made of a nitrocellulose membrane, and then dried at 50° C. for 30 minutes.

After drying, the chromatographic membrane carrier 11 was blocked by dipping in a blocking solution (BSA-containing phosphate buffer, pH 7.0). After blocking, the carrier 11 was washed with a wash (SDS-containing phosphate buffer, pH 7.0) and dried at 40° C. for 120 minutes to prepare the chromatographic membrane carrier 11.

### (3-2) Preparation of a Label Retaining Member 9

Blue colored polystyrene latex particles (particle diameter 0.3 μm) were sensitized with an anti-adenovirus monoclonal antibody (mouse IgG monoclonal antibody recognizing adenovirus at a site different from the site recognized by the antibody used in the judgment part 11E) and then suspended in a dispersing buffer solution (phosphate buffer, pH 7.0, containing BSA and sucrose), to prepare anti-adenovirus monoclonal antibody-sensitized latex particles.

Streptavidin-sensitized latex particles were prepared in the same manner as in (1-2) above.

The anti-adenovirus monoclonal antibody-sensitized latex particles were mixed with the streptavidin-sensitized latex particles, and the resulting mixed latex was added to a glass fiber pad (832 μL/300 mm×5 mm) and then dried in a vacuum drier to prepare a label retaining member 9.

### (3-3) Attachment of Each Member to a Base Material and Cutting of the Base Material

As shown in FIG. 1(c), the chromatographic membrane carrier 11 prepared in (3-1) above, the label retaining member 9 prepared in (3-2) above, a sample addition member 7 consisting of a nonwoven fabric (rayon), and an absorbent member 13 consisting of a nonwoven fabric (cellulose) were attached to a base material 5 consisting of a backing sheet. Then, a transparent sheet 14 was attached so as to cover the sample addition member 7 and absorbent member 13 as shown in the figure. Finally, the specimen was cut into pieces of 5 mm in width by a cutting machine (BioDot Ltd.) to prepare a test device 33 for adenovirus assay.

### (4) Sample Treatment Liquid

0.05 mol of PIPES buffer, pH 7.8 containing 0.1 v/v % nonionic surfactant NP40 (polyoxyethylene (9) octyl phenyl ether), 0.03 w/v % potassium thiocyanate, 0.03 w/v % 2-mercaptoethylamine hydrochloride, 0.5 w/v % EDTA 2 Na and 1.3 w/v % sodium chloride was prepared as a sample treatment liquid. 2.4 ml of this sample treatment liquid was accommodated in a plastic bottle 41 shown in FIG. 3.

### (5) Assay

### (5-1) Examination of Patients Suspected of Having Influenza

Using a suction catheter with a trap, aspirates from the nasal cavity were collected as samples from 133 patients (average age 6.7 years old, from 0.4 to 22 years old) with the maximum body temperature of 38° C. or more who had been suspected of having influenza and had been within 72 hours of the start of symptoms, out of patients who had gone to hospital 1 in a period of from October 2005 to March 2006.

A cotton-tipped swab that had been dipped in the collected sample was placed in the plastic bottle 41 accommodating the sample treatment liquid, and by pinching the bottle 41 with fingers, the sample was extracted into the sample treatment liquid to prepare a measurement sample, and then an opening of the plastic bottle 41 was fitted with a nozzle 42 through which the measurement sample was then introduced into the sample container 1. The test device 31 for influenza virus assay was introduced into the test container 1 accommodating the measurement sample, thereby examining influenza virus. Alternatively, an RNA extracted from the same collected aspirate from the nasal cavity was used in detecting influenza A virus gene, influenza B virus gene and RS virus gene by RT-PCR, to identify the infection-causing virus. The rate of concordance between the assay results by the test device 31 and the detection results by RT-PCR is shown in Table 1.

TABLE 1

| | RT-PCR | | | |
|---|---|---|---|---|
| | Influenza A | | Influenza B | |
| | + | − | + | − |
| Test Device | 85% (61/72) | 97% (59/61) | 67% (4/6) | 100% (127/127) |
| 31 | | | | |

With respect to 3 samples confirmed to be influenza-negative and RS virus-positive by RT-PCR out of the samples suspected of having influenza, the corresponding measurement samples prepared in 5-2 were assayed with the test device 32 for assaying an RS virus infection, and as a result, all the 3 samples were confirmed to be positive. With respect to 11 samples confirmed to be influenza-negative by the test device 31 and RS virus-negative by the test device 32 out of the samples suspected of having influenza, the corresponding measurement samples prepared in 5-1 were assayed with the test device 33 for assaying an adenovirus infection, and as a result, all the 11 samples were confirmed to be positive.

### (5-2) Examination of Patients Suspected of Having RS Virus

Using a suction catheter with a trap, aspirates from the nasal cavity were collected as samples from 102 patients (average age 1.0 year old, from 0.2 to 9 years old) who had been suspected of having RS virus because of fever at a body temperature of 37.5° C. or more or upper respiratory tract symptoms such as runny nose and lower respiratory tract symptoms such as cough and pulmonary auscultation abnormality, out of patients who had gone to hospital 1 in a period of from October 2005 to March 2006.

Using a cotton-tipped swab, fluids wiped out of the nasal cavity were collected as samples from 105 patients (average age 1.5 years old, from 0.2 to 5 years old) who had been suspected of having RS virus because of fever at a body temperature of 37.5° C. or more or upper respiratory tract symptoms such as runny nose and lower respiratory tract symptoms such as cough and pulmonary auscultation abnormality, out of patients who had gone to hospital 2 in a period of from October 2005 to January 2006.

A cotton-tipped swab that had been dipped in the collected sample was placed in the plastic bottle 41 accommodating the sample treatment liquid, and by pinching the bottle 41 with fingers, the sample was extracted into the sample treatment liquid to prepare a measurement sample, and then an opening of the plastic bottle 41 was fitted with a nozzle 42 through which the measurement sample was then introduced into the sample container 1. The test device 32 for RS virus assay was introduced into the test container 1 accommodating the measurement sample, thereby examining RS virus. Alternatively, an RNA extracted from the collected aspirate from the nasal cavity and fluid wiped out of the nasal cavity was used in detecting influenza A virus gene, influenza B virus gene and RS virus gene by RT-PCR, to identify the infection-causing virus. The rate of concordance between the assay results by the test device 32 and the detection results by RT-PCR is shown in Table 2.

TABLE 2

RT-PCR

| | + | − |
|---|---|---|
| Test device 32 for RS virus assay | 82% (99/121) | 97 (83/86) |

With respect to 3 samples confirmed to be RS influenza-negative by RT-PCR out of the samples suspected of having RS virus, the corresponding measurement samples prepared in 5-2 were assayed with the test device 31 for assaying an influenza virus infection, and as a result, all the 3 samples were confirmed to be positive.

What is claimed is:

1. An assay method for virus respiratory infection, comprising steps of:
providing a first immunochromatographic test device for assaying a first virus respiratory infection, and a second immunochromatographic test device for assaying a second virus respiratory infection different from the first virus respiratory infection;
preparing an assay sample by treating a biological sample with a sample treatment liquid;
assaying a part of the assay sample by using the first test device; and
assaying a part of the assay sample by using the second test device when the assay result with the first test device is negative.

2. The assay method of claim 1, wherein the providing step is performed by providing the first test device, the second test device and a third immunochromatographic test device for assaying a third virus respiratory infection different from the first and second virus respiratory infections,
the assay method further comprising a step of assaying a part of the assay sample by using the third test device when the assay result with the second test device is negative.

3. The assay method of claim 1, wherein the assay sample is suitable for multiple virus respiratory infections.

4. The assay method of claim 1, wherein the first and second virus respiratory infections are selected from an influenza virus infection, an adenovirus infection and an RS virus infection.

5. The assay method of claim 1, wherein the first virus respiratory infection is an influenza virus infection and the second virus respiratory infection is an RS virus infection.

6. The assay method of claim 1, wherein the sample treatment liquid is an aqueous solution containing a surfactant.

7. The assay method of claim 6, wherein the sample treatment liquid comprises a reducing agent.

8. The assay method of claim 6, wherein the surfactant is a nonionic surfactant.

9. The assay method of claim 6, wherein the sample treatment liquid comprises a thiocyanic acid compound.

10. The assay method of claim 6, wherein the sample treatment liquid comprises a chelating solution.

11. The assay method of claim 6, wherein the sample treatment liquid comprises a Good buffer.

12. The assay method of claim 1, wherein the first and second test devices are test devices of lateral flow type.

13. The assay method of claim 1, wherein the biological sample is an aspirate from the nasal cavity, a fluid wiped out of the nasal cavity or a fluid wiped out of the pharynx.

14. An assay kit for assaying virus respiratory infection, comprising:
a sample treatment liquid for preparing an assay sample;
a sample treatment container accommodating the sample treatment liquid;
a first immunochromatographic test device for assaying a first virus respiratory infection; and
a second immunochromatographic test device for assaying a second virus respiratory infection different from the first virus respiratory infection.

15. The assay kit of claim 14, wherein the sample treatment container comprises a bottle having an opening and a cap for closing the opening.

16. The assay kit of claim 15, further comprising a nozzle that can be fitted into the opening of the bottle.

17. The assay kit of claim 16, wherein the nozzle comprises a filter therein.

18. The assay kit of claim 14, wherein the sample treatment liquid is an aqueous solution containing a surfactant.

19. The assay kit of claim 18, wherein the sample treatment liquid comprises a thiocyanic acid compound.

20. The assay kit of claim 14, wherein the first and second test devices are test devices of lateral flow type.
